(19) United States
(12) Patent Application Publication
(10) Pub. No.: US 2003/0175831 A1
(43) Pub. Date: Sep. 18, 2003

(54) Monoreagent for assaying platelet-derived microparticles

(76) Inventors: Michel Canton, Marseille (FR); Isabelle Botosezzy, Marseille (FR)

Correspondence Address: FOLEY AND LARDNER, SUITE 500, 3000 K STREET NW, WASHINGTON, DC, 20007, US

(21) Appl. No.: 10/276,609

(22) PCT Filed: May 15, 2001
(86) PCT No.: PCT/FR01/01468

(30) Foreign Application Priority Data
May 16, 2000 (FR) 00/06247

(51) Int. Cl. G01N 33/567; C12M 1/34

(52) U.S. Cl. 435/7.21; 435/287.2

(57) ABSTRACT

The invention concerns a monoreagent and a diagnostic kit for detecting and quantifying platelet-derived microparticles (MPP). Said monoreagent, combining several populations of calibrated beads and a double labelling of MPP, enables both an optimal isolation of MPP based on a size criterion, their characterisation by specific labels, and their numbering with counting beads.

The present invention relates to a monoreagent, and to a diagnostic kit comprising it, for detecting and quantifying platelet-derived microparticles (PMPs) by double color labeling.

Activated platelets are known to release vesicles called platelet-derived microparticles (PMPs). These particles, which are small in diameter (possibly ranging from 0.1 to 0.8 μm however), result from vesiculation of the platelet plasma membrane after certain stimuli (thrombin, collagen, C5b9, etc.). By virtue of their prothrombotic and procoagulant activity, PMPs appear to play an important role in vivo.

PMPs express several platelet receptors (GpIb, GpIIb/IIIa, etc.) and are capable of binding several types of platelet ligand (fibrinogen, fibronectin, collagen, vWF, vitronectin, etc.) and of adhering to the subendothelial matrix, at a vascular opening. High circulating PMP levels have been observed in many pathologies, such as: unstable angina, myocardial infarction, coronary angiography, diabetes mellitus, cardiopulmonary shunt, paroxysmal nocturnal hemoglobinurea, aplastic anemia, HIT (heparin-induced thrombocytopenia), idiopathic thrombocytopenic purpura, etc. Conversely, some pathologies, such as Scott syndrome, may be associated with a deficiency in circulating PMP level.

Thus, detection and counting of these PMPs appears to be an important criterion in detecting and evaluating the severity of a prothrombotic condition in the course of various pathologies.

The phenomena associated with the formation of platelet-derived microparticles and the value of detecting them for diagnosis have been widely described in the literature (1-5).

Various approaches based on techniques such as, for example, filtration (6), ELISA (7) or, more particularly, flow cytometry (8-11) have thus been proposed for isolating and/or quantifying them.

The principle of flow cytometry, and its advantages for discriminating and quantifying subpopulations of cells and cell particles, are today well known to those skilled in the art.

This method has already been used to detect activated platelets in blood samples and to determine procoagulant platelet-derived microparticles using functional assays (12).

However, the studies carried out mostly use single color fluorescent labeling, and/or employ counting beads, the effectiveness of which in terms of calibration is insufficient to envision an. industrializable method for carrying out tests for detecting and quantifying PMPs in a completely standardized manner.

The present invention introduces an improvement into the preexisting methods by providing a new solution which makes it possible, in one step, and in a standardized manner, to specifically detect and count platelet-derived microparticles in a blood sample, without prior treatment.

To this effect, a particular monoreagent, combining several populations of calibrated beads and double labeling of PMPs, has been developed. Thus, such a reagent allows equally 1) optimal isolation of PMPs according to a size criterion, 2) characterization thereof by specific labeling, and 3) counting thereof using counting beads. It therefore offers a means of specifically detecting and counting, very reliably, the number of PMPs in a sample, even when these PMPs are present in a very small amount. The subject of the present invention is thus a monoreagent for detecting and quantifying platelet- derived microparticles (PMPs) in a blood sample by flow cytometry, comprising:

a) a reagent 1 for double labeling the PMPs, comprising:

either annexin V, or any other marker specific for membrane phospholipids, coupled to a fluorochrome 1,

or a population 1 of monoclonal antibodies (MABs), directed against platelet membrane structures, labeled with a fluorochrome 1, (reagent 1a), and

a population 2 of MABs, directed against platelet membrane structures, labeled with a fluorochrome 2 (reagent 1b), said fluorochrome 2 being different from fluorochrome 1, and

b) a reagent 2 consisting of a mixture of microspheres comprising:

a population of microspheres A labeled with one of the two fluorochromes 1 and 2, or another fluorochrome with a spectrum similar to one of the two, used to define the region of analysis of the PMPs in terms of size (light scattering parameters) and allowing differentiation thereof (setting beads),

a population of microspheres B, unlabeled or labeled with one of the two fluorochromes 1 and 2, or another fluorochrome with a spectrum similar to one of the two, the concentration of which is known, used as internal standard for counting the microparticles (counting beads),

a population of microspheres C labeled only with fluorochrome 1, used to define, with respect to the fluorochrome 1 fluorescence parameter, the minimum intensity threshold which delimits the region of analysis of the microparticles to which the MABs 1 or the annexin V, or any other marker specific for membrane phospholipids labeled with fluorochrome 1, are bound (threshold beads),

and a population of microspheres D, of diameter identical to C, and labeled only with fluorochrome 2, used to define, with respect to the fluorochrome 2 fluorescence parameter, the minimum intensity threshold which delimits the region of analysis of the microparticles to which the MABs 2, labeled with fluorochrome 2, are bound (threshold beads).

The monoreagent according to the invention therefore has several functions, which are:

double color labeling of the microparticles, allowing optimal differentiation between the platelet-derived microparticles and the equivalents of another origin,

standardizing the thresholds for discriminating between microparticles of interest and the other particles and debris responsible for a possible background noise,

standardizing the counting of the labeled platelet- derived microparticles which are discriminated via the reference counting of the internal standard bead.

Advantageously, each population C and D of threshold beads is composed of two subpopulations of beads with 2 levels of fluorescence 1 or 2, so as to optimize the standardization of the analysis by setting the thresholds and adjusting the compensations.

The various populations of beads can be obtained from diverse materials conventionally used for producing this type of particle. They are, for example, organic polymers, such as polysaccharides, styrene polymers, polyacrylates, pblyacrylamide, poly(hydroxyethyl methacrylate), polyvinyls, polystyrenes and polymers containing aromatic groups. A material preferentially used is polystyrene. The material may, however, be different between the various populations of beads used.

Several fluorochromes are commercially available. Phycoerythrin (PE) or fluorescein, such as, for example, fluorescein isothiocyanate (FITC), is advantageously used.

The counting beads, the role of which is to count the PMPs, are advantageously greater than or equal to 5 μm in diameter, so that, in cytometry, the cloud of counting beads is quite distinct from cells and from beads of another population (threshold beads). Preferentially, they are between 5 and 10 μm in diameter.

These counting beads may be unlabeled or labeled, within the mass or at the surface. They are preferentially labeled in the mass with one of fluorochromes 1 and 2 used for labeling the threshold beads, or another fluorochrome with a spectrum similar to one of them.

The setting beads make it possible to set the analysis window for the PMPs, according to size. They are advantageously between 1 and 0.5 μm in diameter. They are labeled with one of the two fluorochromes 1 and 2, or another fluorochrome with a spectrum similar to one of the two, within the mass or at the surface.

The role of the threshold beads is to standardize the analysis, by setting the analysis window for the PMPs, and to allow the compensations to be adjusted. They are between 1 and 5 μm, and preferentially 3 μm, in diameter.

The two populations C and D of threshold beads consist of two populations labeled with one of fluorochromes 1 and 2 and advantageously both consist of two categories of beads of the same nature but having different fluorescence levels.

These calibrants are advantageously standardized as MESF (Molecules of Equivalent Soluble Fluorochrome—13) using a standard (Quantum 24 fluorescein—Code: RF 824; Quantum PE R-Phycoerythrin—Code RF 827, FCSC Europe).

The intensity of the threshold beads is thus set so as to be at the limit between the minimum intensity of the PMPs and maximum intensity of the microparticles of other origin and of the contaminants of similar size (dust, cell debris, etc.).

The value of the fluorochrome 1 and fluorochrome 2, preferentially FITC and PE, thresholds is determined on a group of normal and pathological individuals using commercial standard calibrants, for optimal discrimination of the PMPs relative to the other contaminants.

The value of the thresholds thus defined gives the value of the associated threshold beads (C, D) of the monoreagent.

The monoclonal antibodies (MABs) of the populations 1 and 2 are advantageously directed against platelet membrane structures chosen from the following specificities: CD61, CD41, CD42a, CD42b, CD42c, CD49b, CD29, CD62P, CD63, protein S and prothrombin.

These populations 1 and 2 can be represented by a single MAB, by several MABs with the same specificity but directed against different epitopes, or by several MABs with different platelet specificities.

According to a preferred variant, reagent 1 of the monoreagent of the invention consists of annexin V, or another marker specific for membrane phospholipids, labeled with a fluorochrome 1 (reagent 1a), and of a population 2 of MABs labeled with a fluorochrome 2 (reagent 1b).

Use is preferably made of a population 2 of MABs comprising several MABs with different specificities, and more preferentially anti-CD61 MABs and anti-CD42b MABS. An example of a preferred reagent consists of anti-CD61 and anti-CD42b MABs labeled with PE, and annexin V labeled with FITC.

According to an advantageous variant, the anti-CD61 antibodies are produced by the P18 and 4F8 hybridomas deposited with the BCCM/LMBP Collection (Belgian Coordinated Collections of Microorganisms) according to the Treaty of Budapest, on 06.26.97 and 06.02.98, respectively, under the Nos LMBP162CB (P18) and LMBPI667CB (4F8).

Combination of these two MABs is particularly advantageous since there is no steric gene between them, and the binding thereof to the PMPs is not impaired by the possible presence of commercial anti-GPIIb/IIIa anti-aggregating agents such as Reopro (Abciximab, Centocor), Integrilin (Ceptifibatide, Shering-Plough) or Agrastat (Tirofiban, Merck).

The anti-CD42b MABs are, for example, represented by the antibody SZ2. This can be obtained from the company Beckman Coulter/Immunotech (Ref. IM0409).

Annexin V is commercially available. It may, for example, come from Bender (Ref. BMS306 FI).

Other compounds which can be used as markers specific for membrane phospholipids are, for example, anti-phosphatidylserine antibodies, such as the antibodies BA3B5C4 and 3SB9b described in the literature (14) or the Ab-1 antibodies distributed by France Biochem (Ref. AM31, Oncogene Research Product).

Advantageously, the sample consists of whole blood taken on CTAD (citrate, theophylline, adenosine, dipyridamole), sodium citrate or EDTA. It may also consist of plasma, although this type of sample, because of the prior preparation step which it requires, is not preferentially used.

According to a second aspect, the invention relates to a diagnostic kit comprising a monoreagent as mentioned above, intended for detecting and quantifying PMPs in a blood sample.

Such a diagnostic kit thus advantageously comprises:

a reagent 1a consisting of a population 1 of MABs specific for platelet membrane structures, or annexin V or any other marker specific for membrane phospholipids, labeled with a fluorochrome 1,

a reagent 1b consisting of a population 2 of MABs specific for platelet membrane structures, labeled with a fluorochrome 2, said fluorochrome 2 being different from fluorochrome 1,

a reagent 2 consisting of:

a population of beads for setting the analysis window for the PMPs, labeled with fluorochrome 1 or 2, or another fluorochrome with a spectrum similar to one of the two,

a population of counting beads, unlabeled or labeled with fluorochrome 1 or 2, or another fluorochrome with a spectrum similar to one of the two,

a population of threshold beads, preferentially consisting of two subpopulations of beads with two levels, labeled with fluorochrome 1,

a population of threshold beads, preferentially consisting of two subpopulations of beads with two levels, labeled with fluorochrome 2, and preferably,

a reagent 3 consisting of a dilution buffer.

Preferentially, reagents 1, 2 and 3 are mixed.

According to an advantageous variant, reagent 1a consists of annexin V. In this case, the buffer for diluting reagent 3 is a calcium buffer, for example consisting of a Hepes/NaCl/CaCl₂ mixture, since the binding of annexin to phospholipids is calcium-dependent (15). In this case, the use of a sample treated with EDTA will, of course, be avoided.

Reagent 1b is advantageously composed of a mixture of MABs with different specificities, and preferentially anti-CD61 and anti-CD42b.

More particularly, reagent 1b consists of a mixture of MABs P18 and 4F8 (anti-CD61) and SZ2 (anti-CD42b).

FITC is preferably used as fluorochrome 1 and PE as fluorochrome 2.

To ensure good conservation, all the reagents of the kit of the invention may contain 0.09% (0.09 g/l) sodium azide.

According to another additional aspect, a subject of the invention is a method for detecting and quantifying PMPs, characterized in that it comprises the following steps:

a) bringing a blood sample into contact with a monoreagent as defined above and incubating them, so as to obtain double color labeling of said PMPs, and

b) cytometrically analyzing the double labeled events.

Finally, the invention is directed toward the use of a monoreagent as defined above, or of a diagnostic kit comprising said monoreagent, in a method for detecting and monitoring a prothrombotic condition.

The principle of the method of cytometric analysis used in the context of the invention is recalled below:

Cytometric analysis:

Reference should be made to the operator's manual for the apparatus, provided by the manufacturer, for instructions on how to perform cytometric readings. Before analysis, the tubes are homogenized using a Vortex mixer.

For carrying out the protocol, the following are necessary:

1 FS×SS cytogram

2 FL1 LOG histograms, one gated by the FS×SS cytogram (for the setting beads A) and the other gated by the FS×SS cytogram (for the counting beads B)

2 FL1 LOG/FL2 LOG cytograms, one cytogram gated on the threshold beads (C and D) and the other gated on the beads A+PMPs.

An FS LOG×SS LOG cytogram is constructed. A discriminating threshold is set to eliminate possible contaminants (background noise of the apparatus). 3 analysis windows are drawn around the various populations of beads:

Window R1 for the 0.8 μm setting beads

Window R2 for the 5 to 10 μm counting beads

Window R3 for the 3 μm threshold beads.

2 FL1 LOG histograms are created, one for the setting beads (gated by the window R1), the other for the counting beads (gated by the window R2).

2 FL1 LOG/FL2 LOG cytograms are created, one cytogram for the threshold beads C and/or D (gated by the window R3) and the other for the PMPs (gated by the window R1).

For optimal analysis conditions, the FL1 and FL2 photomultiplier voltage is adjusted such that the upper threshold bead (C or D) is set at the beginning of the 4^{th} decade in FL1 or FL2, respectively.

The compensations are set up on the threshold beads (C and D).

Set Up of the FL2 Compensation

On the FL1 LOG/FL2 LOG cytogram gated on the “R3” analysis window for the beads D, 2 windows are set (R4 and R5) on the clouds corresponding, respectively, to the upper bead and to the lower bead. The FL2 fluorescence is compensated (FL2 −x% FL1) until equivalence of the FL2 LOG mean fluorescence intensity (MFI) of the R4 and R5 windows.

Set Up of the FL1 Compensation

The FL1 LOG and FL2 LOG fluorescence settings (FL1 and FL2 photomultiplier, PMT, voltages) previously set are not changed for the rest of the protocol.

The procedure is the same as that described above. On the FL1 LOG/FL2 LOG cytogram gated on the “R3” analysis window for the beads C, 2 windows (R6 and R7) are set on the clouds corresponding, respectively, to the upper bead and to the lower bead. The FL1 fluorescence is compensated (FL1 −x% FL2) until equivalence of the FL1 LOG MFI of the R6 and R7 windows.

The examples below illustrate the present invention.

### EXAMPLE NO. 1

### Study of the Threshold Beads of a Monoreagent According to the Invention

The monoreagent illustrated below is based on double labeling of PMPs with annexin V-FITC and CD 41-PE.

1.1 Preparation of Threshold Beads

The threshold beads were prepared from 3 μm polystyrene beads coated with different amounts of a murine IgG which does not react with the blood elements mentioned. The murine IgG load is chosen according to the level of fluorescence intensity desired for this threshold bead. The beads are fluorescence labeled by contact with an anti-murine IgG reagent corresponding either to:

FITC labeled sheep F(ab′)2 anti-mouse IgG (H+L), human absorbed

FITC labeled goat Fab anti-mouse IgG (H+L), human absorbed

PE labeled sheep F(ab′)2 anti-mouse IgG (H+L), human absorbed

PE labeled goat Fab anti-mouse IgG (H+L), human absorbed

a. Preparation of IgG-coated Beads

The batch of beads is chosen to give a mean fluorescence intensity which is shifted compared to a population of unlabeled platelets (for example, mean fluorescence of the beads 10 times greater than the mean fluorescence of the platelets).

Materials

MAb Sendo-3, anti-CD146, clone F439-E10, which does not react with the blood elements mentioned (Leukocyte Typing VI, Kishimoto, Kikutani et al.)

L300 beads (Estapor)

Buffers (Table 1):

| BUFFERS | SOLUTIONS |
|---|---|
| Adsorption | PBS |
| Saturation | PBS, 4% BSA |
| Washing | PBS |
| Fixing | PBS, 1% PFA, 0.09% azide |
| Storage | PBS, 0.1% BSA; 0.09% azide |
| Dilution | PBS |

Methods

Preparation of Buffers

1 liter of PBS is prepared and filtered through a 0.22 μm membrane with a 500 ml filtration system. 1 liter of PBS, 1% PFA, 0.09% sodium azide is prepared. 1 liter of PBS BA is prepared and filtered through a 0.22 μm membrane with a 500 ml filtration system. NB: All the buffers are filtered through a 0.22 μm membrane before use.

Preparation of Coupling Reagents

a) Dilution of IgG1s

The various levels of bead loading are obtained by varying the IgG1 concentration of the coating solutions.

1. An aliquot of Sendo3 at 5 mg/ml is thawed.

2. A solution of Sendo3 at 500 μg/ml (1/10 dilution) is prepared as follows:

100 μl of Sendo3 at 5 mg/ml are placed in a CMF tube

900 μl of PBS are added to this same tube.

3. A solution of Sendo3 at 100 μl /ml (⅕ dilution) is prepared as follows:

200 μl of Sendo3 at 500 μg/ml are placed in a CMF tube

800 μl of PBS are added to this same tube.

4. The 2 coating solutions are prepared as indicated in Table 2:

| | SE3 | | Final IgG1 |
|---|---|---|---|
| | 100 μg/ml | PBS | concentration |
| Bead C | 250 μl | 4.75 ml | 5 μg/ml |
| Bead D | 750 μl | 4.25 ml | 15 μg/ml |

b) Preparation of Microspheres Before Coupling

General Microsphere Washing Protocol

The washes and buffer changes are all performed as follows:

1. The beads are centrifuged at 3000 rpm for 8 min (1890 g, 8160 rotor), with the brake on.

2. The supernatant is removed with a liquid jet vacuum pump.

3. The resuspension buffer is added with a pipette.

4. The mixture is thoroughly vortexed in order to resuspend the pellet.

Washing of Beads Before Coupling

1. 6 ml of 10% bead suspension (verified at approximately 4 106 microspheres/μl) are deposited.

2. The suspension is centrifuged and the supernatant is removed.

3. The pellet is resuspended with 7.5 ml of PBS.

4. The suspension is centrifuged and the supernatant is removed.

5. The pellet is resuspended with 6 ml of PBS.

Coupling Protocol

a. The tube containing the washed microspheres is vortexed.

b. The tube containing the coating solution is vortexed.

c. The tube containing the coating solution is continually mixed on the vortex. 1 ml of washed microspheres is rapidly added with a 1 ml Gilson, in a single step, vertically at the center of the solution.

d. The tube is stoppered.

e. The tube is placed on a rotary mixer at +2-8° C.

f. Steps b to e are repeated with the other coating solutions.

g. The mixture is incubated overnight with mixing.

h. The saturation buffer is prepared (2 g of BSA A7030, qs 50 ml of PBS), and filtered through a 0.22 μm filter.

i. 100 ml of fixing buffer are filtered through a 0.22 μm membrane with a syringe filter.

j. After centrifugation, the supernatants are removed (see general microsphere washing protocol).

k. The pellets are taken up with 5 ml of saturation buffer and vortexed.

l. After centrifugation, the supernatants are removed.

m. The pellets are taken up with 10 ml of PBS and vortexed.

n. After centrifugation, the supernatants are removed.

o. The pellets are taken up with 10 ml of fixing buffer and vortexed.

p. The mixture is incubated for 10 min at ambient temperature with mixing.

q. After centrifugation, the supernatants are removed.

r. The pellets are taken up with 10 ml of PBS-BA and vortexed.

s. After centrifugation, the supernatants are removed.

t. The pellets are taken up with 10 ml of PBS-BA and vortexed.

u. After centrifugation, the supernatants are removed.

v. The pellets are taken up with 7.5 ml of PBS-BA and vortexed.

The individual suspensions (solution at approximately 500,000 microspheres/μl) are ready and are stored at +2-8° C.

b. Preparation of Fluorescent Threshold Beads Using F(ab′)2s Conjugated to FITC or to PE

Materials

anti-mouse sheep immunoglobulin F(ab′)2s coupled to FITC (Silenus, ref. DDAF)

anti-mouse sheep immunoglobulin F(ab′)2s coupled to PE (Silenus, ref. DDAPE)

Methods

1. 2.5 ml of Sendo-3-coated L300 beads (Estapor) (300,000 microspheres/μl) are pipetted

Sendo-3 at 15 μg/ml for the FITC bead

Sendo-3 at 5 μg/ml for the PE bead.

2. The beads are centrifuged at 3000 rpm for 8 minutes. The supernatant is removed.

3. The beads are taken up in 500 μl of 1×PBS BA buffer.

4. 12.5 ml of DDAF or DDAPE, diluted to 1/100 in 1×PBS BA, are added to the bead preparation. The mixture is homogenized.

5. The mixture is incubated for 1 hour at, ambient temperature with rotary mixing.

6. 2 washes are performed as follows:

25 ml of 1×PBS BA are added

The mixture is centrifuged at 3000 rpm for 8 minutes.

The supernatant is removed.

7. 2.5 ml of a solution of mouse gamma globulin (Jackson, #015-000-002) at 100 μg/ml are added to the bead pellet in order to neutralize the free anti-mouse IgG sites of the DDAF and DDAPE reagents.

8. The mixture is incubated for 1 hour at ambient temperature with rotary mixing.

9. 1 wash is performed as described above.

10. The bead pellet is taken up in 2.5 ml of 1X PBS BA.

c. Preparation of Threshold Beads Using Fabs Conjugated to FITC or to PE

Materials

Anti-mouse goat immunoglobulin Fabs coupled to FITC (Protos, ref.#341)

Anti-mouse goat immunoglobulin Fabs coupled to PE (Protos, ref.#441).

Method

As above, without steps 7, 8 and 9.

1.2 Cytometric Analysis

The 2 types of FITC threshold bead and PE threshold bead prepared were tested alone in PBS-BA buffer or in the presence of all the other reagents making up the monoreagent (MAbs coupled to FITC or to PE and 0.85 μm setting beads A). The scatter parameters (forward-angle scatter herein referred to as FS, side-angle scatter herein referred to as SS) are analyzed on a logarithmic scale, with a discriminator set on SS.

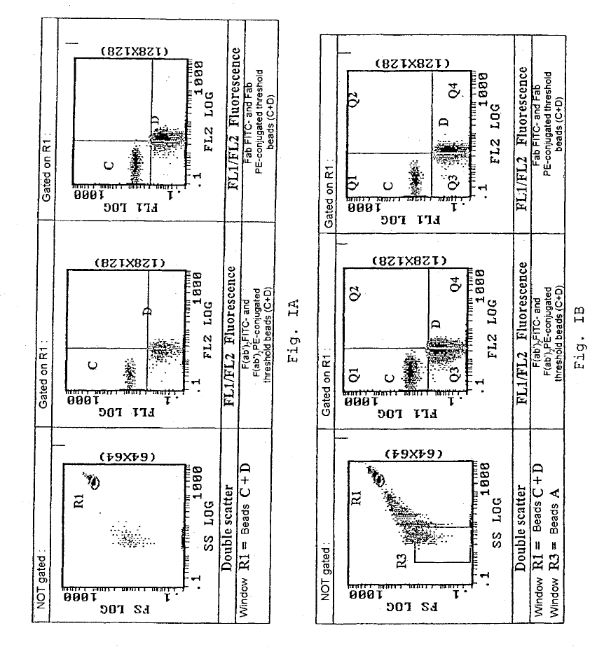

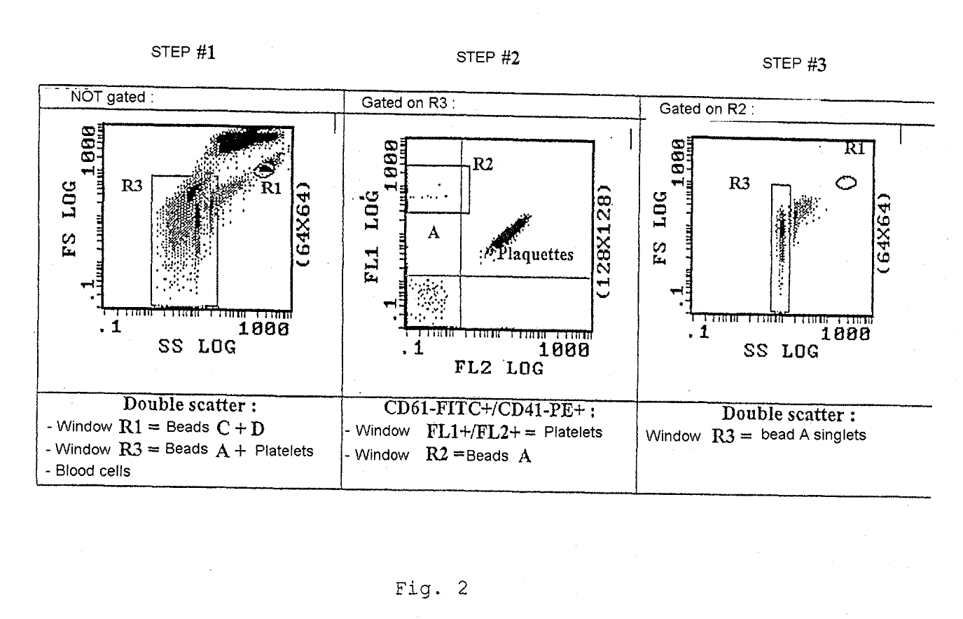

The results are given in FIG. 1, in which:

FIG. 1a) represents the analysis of the threshold beads in PBS BA buffer;

FIG. 1b) represents the analysis of the threshold beads with the beads A in the monoreagent.

In these figures, the R1 window corresponds to the 3 μm FITC threshold beads and PE threshold beads, the R3 window corresponds to the double scatter PMP analysis window, the beads D correspond to the FITC threshold beads and the beads C correspond to the PE threshold beads.

1.3 Conclusion

It is possible to use the mixture of 3 μm beads labeled with FITC and with PE as described to set an analysis window (herein referred to as Q2) for double labeled elements. In the example, the threshold on FL2 log (vertical threshold) was chosen at the level of the mean value of the beads D, and the threshold on FL1 log (horizontal threshold) was chosen to be equal to 1/3.5 of the mean value of the beads C.

The functionality of an extemporaneous combination of the various categories of beads (3 μm FITC threshold beads and PE threshold beads for setting a region of measurement Q2 and 0.85 μm FITC beads for setting the R3 analysis window for the PMPs) required for carrying out the test as a monoreagent is demonstrated.

### EXAMPLE NO. 2

### Test of the Monoreagent According to the Invention

2.1 Materials

CD61: P18-FITC (purified and labeled according to standard procedures known to those skilled in the art) (reagent filtered through 0.1 μm)

P18 (McGregor J L, Thromb.Haemost. 1987, 58, 507)

CD41a: P2-PE (ref. IM1416, Immunotech)

P2 (ref. IM0145, Immunotech)

Annexin V FITC (ref. BMS306FI/a, Bender Medsystems)

0.85 μm setting beads (A) (Cat. VFP 08525 Avidin coated Yellow, Spherotec)

F(ab′)2 FITC- and F(ab′)2 PE-conjugated threshold beads (C and D) described above

1×CaCl2 buffer (10 mM Hepes, 140 mM NaCl, 2.5 mM CaCl2)

CaCl₂/EDTA buffer (10 mM Hepes, 140 mM NaCl, 2.5 mM CaCl₂, 6.2 mM EDTA, pH 7.4)

2.2 Protocol

1. 30 μl of whole blood taken on sodium citrate or EDTA and diluted to 1/10 in 1×PBS BA are pipetted.

2. 10 μl of noncoupled MAbs or buffer (1×PBS BA or CaCl₂ or CaCl₂/EDTA) are pipetted.

3. Incubation is carried out for 10 minutes at ambient temperature.

4. 10 μl of MAb coupled to FITC or annexin V coupled to FITC are added.

5. 10 μl of MAb coupled to PE are pipetted.

6. 5 μl of FITC threshold beads (200,000 microspheres/μl) are pipetted.

7. 5 μl of PE threshold beads (200,000 microspheres/μl) are pipetted.

8. 5 μl of 0.85 μm Spherotec beads diluted to 1/20 in 1×PBS BA are pipetted.

9. Incubation is carried out for 20 minutes at room temperature.

10. 1 ml of buffer (1×PBS BA or CaCl₂ or CaCl₂/EDTA) is added.

11. Cytometric analysis: analysis time of 2 minutes, slow flow rate.

Comment:

1. In the context of these monoreagent tests, the threshold beads act as beads for counting the PMPs.

2. In the event of inhibition of fixing of the MAbs coupled to the fluorochromes, a 10 minute preincubation of the blood sample with the noncoupled MAbs, at ambient temperature, is carried out.

2.3 Operating Conditions

| | TESTS | | | | |
|---|---|---|---|---|---|
| REAGENTS | A | B | C | D | E |
| P18-FITC at 20 μg/ml (5 μg/ml final) | X | | X | X | X |
| P2-PE Immunotech | X | X | X | X | |
| P2 at 100 μg/ml (20 μg/ml final) | | | X | | |
| P18 at 100 μg/ml (20 μg/ml final) | | | | X | |
| Annexin V-FITC diluted to 1/40 in CaCl₂ | | X | | | |
| Annexin V-FITC diluted to 1/40 in CaCl₂/EDTA | | | | | X |
| PBS BA buffer | X | | X | X | |
| CaCl₂ buffer | | X | | | |
| CaCl₂/EDTA buffer | | | | | X |

Test A makes it possible to count the PMPs and the platelets less than or equal to 0.85 μm in size and corresponding to CD61+/CD41a+ events.

Test B makes it possible to count the activated PMPs and/or the activated platelets less than or equal to 0.85 μm in size corresponding to annexin V+/CD41a+ events. Only this test makes it possible to demonstrate the platelet-derived elements from the blood which are less than or equal to 0.85 μm in size and have an activated phenotype (by virtue of the presence, on the outer cell membrane of the platelets and of the PMPs, of phosphatidylserine residues revealed by the annexin V).

Tests C, D and E make it possible to establish the specificity of the CD41a, CD61 and annexin V reagents, respectively.

2.4 Test of the Monoreagent in Whole Blood

a) Principle of Setting the R3 Window for Double Scatter Analysis of PMPs in Whole Blood

The example below illustrates the principle of double scatter analysis of PMPs in whole blood. The blood sample corresponds to test A (CD61-FITC/CD41-PE).

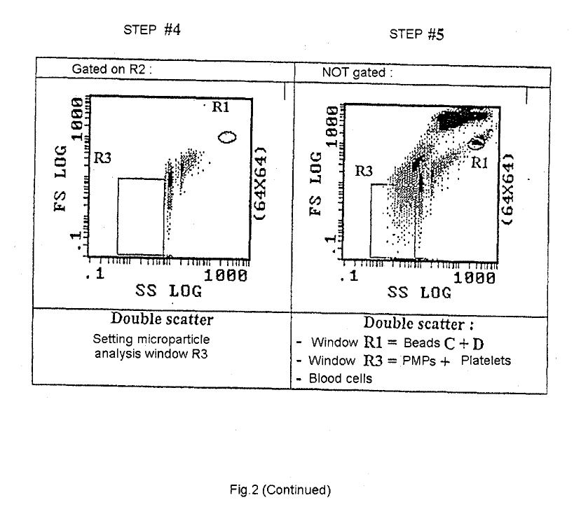

The various steps of the cytometric analysis are given in FIG. 2.

The PMP analysis window R3 is gated using the singlets for 0.85 μm setting beads, FITC-fluorescent in the mass (beads A). A large double scatter window R3 is first of all gated around these beads (STEP #1). An FL1-LOG/FL2-LOG cytogram gated on R3 makes it possible to locate these setting beads in FL1 with a window R2 (STEP #2). A window R3 is then redefined in double scatter and the mean value of the FS Log parameter is measured on this bead A singlet population (STEP #3).

The platelet-derived microparticle analysis window R3 can then be set in double scatter according to the following criteria (STEP #4):

FS-LOG upper limit: equal to the mean value of the FS LOG parameter for the bead A singlet population, measured in the preceding step.

FS-LOG lower limit: do not take the first channel of the FS LOG parameter.

SS-LOG upper limit: against the bead A singlet cloud

SS-LOG lower limit: discriminator on the SS LOG parameter set to the minimum.

Finally, the double scatter analysis of samples (whole blood+monoreagent) with the various cytometric analysis windows can be carried out (STEP #5).

b. Sample Analyses

The test for the functionality of the monoreagent and for the specificity of its components was carried out on 19 whole bloods: 11 bloods taken on EDTA and 7 bloods taken on sodium citrate.

Tests A, B, C, D and E were carried out according to the protocol described above, using blood diluted to 1/10.

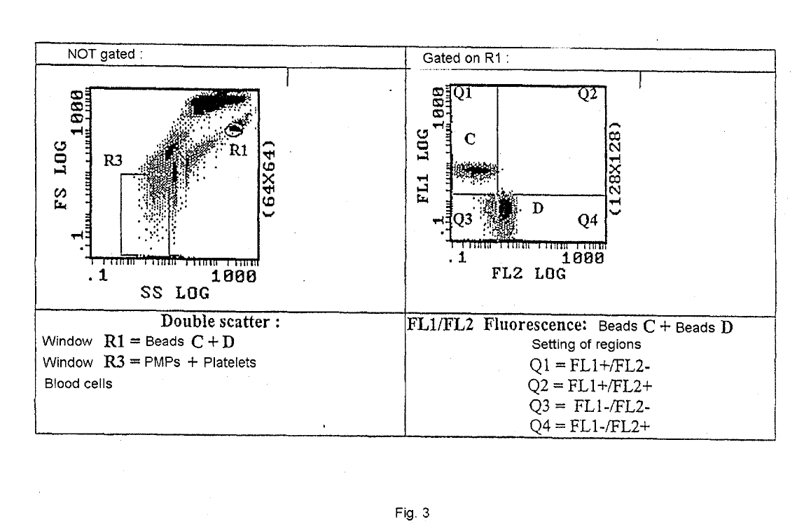

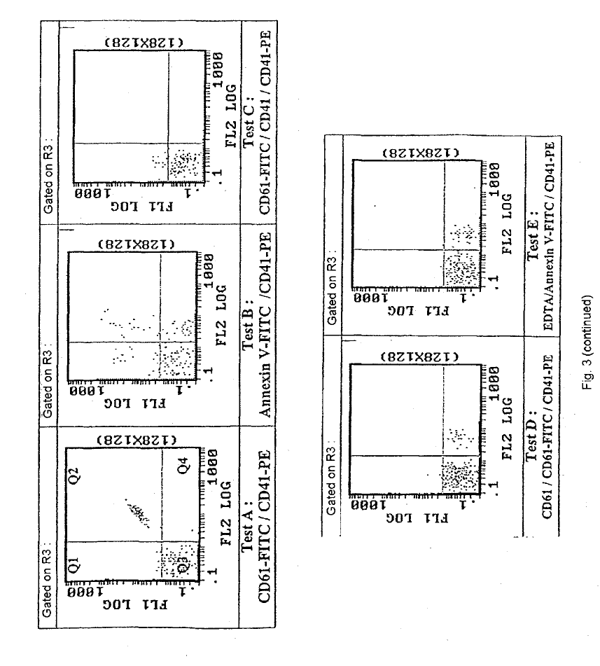

Cytometric results are given in FIG. 3.

Quantitative results are expressed as number of events per μl of whole blood.

The analysis is carried out using the elements counted in the window Q2.

Anticoagulant EDTA:

| Sample | Test A | Test B |
|---|---|---|
| 1 | 6113 | 1094 |
| 2 | 6030 | 1540 |
| 3 | 6776 | 1628 |
| 4 | 9060 | 1115 |
| 5 | 7428 | 974 |
| 6 | 5158 | 1822 |
| 7 | 3131 | 1636 |
| 8 | 4401 | 1189 |
| 9 | 5777 | 666 |
| 10 | 12981 | 756 |
| 11 | 7105 | 504 |
| Mean | 6724 | 1175 |
| Standard deviation | 2603 | 436 |

Anticoagulant Sodium Citrate:

| Sample | Test A | Test B |
|---|---|---|
| 12 | 7002 | 716 |
| 5 | 5610 | 879 |
| 13 | 3205 | 400 |
| 9 | 8095 | 1190 |
| 10 | 10680 | 781 |
| 14 | 5344 | 960 |
| 15 | 13620 | 896 |
| Mean | 7651 | 832 |
| Standard deviation | 3528 | 243 |

Inhibition Tests (Anticoagulant EDTA):

| | | | Percentage inhibition | | |
|---|---|---|---|---|---|
| Sample | Test A | Test B | Test C | Test D | Test E |
| 2 | 6030 | 1540 | 99.32% | 99.25% | 97.01% |
| 3 | 6776 | 1628 | 98.86% | 98.27% | 94.35% |
| 4 | 9060 | 1115 | 98.45% | 98.65% | 92.56% |
| 6 | 5158 | 1822 | 96.76% | 93.68% | 98.30% |
| 7 | 3131 | 1636 | 95.02% | 93.42% | 93.46% |
| 8 | 4401 | 1189 | 96.68% | 94.75% | 95.63% |

### REFERENCES

1. Assembly of the Platelet Prothrombinase Complex Is Linked to Vesiculation of the Platelet Plasma Membrane. Studies in Scott syndrome: an isolated defect in platelet procoagulant activity. P. J. Sims, T. Wiedmer, C. T. Esmon, H. J. Weiss and S. J. Shattil. J. Biol. Chem., vol. 264, No. 29, 17049-17057, 1989.

2. Microparticle generation during in vitro platelet activation by anti-CD9 murine monoclonal antibodies. S. Nomura, H. Nagata, M. Suzuki, K. Kondo, S. Ohga, T. Kawakatsu, H. Kido, T. Fukuroi, K. Yamaguchi, K. Twata, M. Yanabu, T. Soga, T. Kokawa and K. Yasunaga. Thrombosis Research, 62, 429-439, 1991.

3. Platelet microparticles Bind, Activate and Aggregate Neutrophils In Vitro. W. Jy, W.-W. Mao, L. L. Horstman, J. Tao, Y. S. Ahn. Blood Cells, Molecules and Diseases. 21, 217-231, 1995.

4. A new lupus anticoagulant neutralization test based on platelet-derived vesicles. J. Arnout, E. Huybrechts, M. Vanrusselt and J. Vermylen. British Journal of Haematology. 80, 341-346, 1992.

5. Platelet procoagulant activity and microvesicle formation. Its putative role in hemostasis and thrombosis. R. F. A. Zwaal, P. Comfurius and E. M. Bevers. Biochimica et Biophysica Acta. 1180, 1-8, 1992.

6. Demonstration of Platelet-Derived Microvesicles in Blood from Patients with Activated Coagulation and Fibrinolysis Using a Filtration Technique and Western Blotting. P. A. Holme, N. O. Solum, F. Brosstad, M. Roger. Thrombosis and Hemostasis. 72 (5), 666-71, 1994.

7. WO 96/03655 (Feb. 8, 1996)—Inventors: J. M. Freyssinet, B. Antoni, F. Donie, H. Lill.

8. Direct Detection of Activated Platelets and Platelet-Derived Microparticles in Humans. C. S. Abrams, N. Ellison, A. Z. Budzyndki and S. J. Shattil. Blood, vol. 75, No. 1, 128-138, 1990.

9. Annexin V as a Probe of Aminophopholipid Exposure and Platelet Membrane Vesiculation: A flow Cytometry Study Showing a Role for Free Sulfhydryl Groups. J. Dachary-Prigent, J.-M. Freyssinet, J.-M. Pasquet, J.-C. Carron and A. T. Nurden. Blood, vol. 81, No. 10, 2554-2565, 1993.

10. Platelet-derived Microvesicles in Thrombotic Thrombocytopenic Purpura and Hemolytic Uremic Syndrome. M. Galli, A. Grassi, T. Barbui. Thrombosis and Hemostasis. 75 (3), 427-31, 1996.

11. A New Flow Cytometry Method of Platelet-derived Microvesicle Quantification in Plasma. V. Combes, F. Dignat-George, M. Mutin, J. Sampol. Thrombosis and Hemostasis. 77 1, 212-24, 1997.

12. WO 96/15449 (May 23, 1996)—Inventors A. D. Michelson, M. R. Barnard.

13. Terminology and Nomenclature for Standardization in Quantitative Fluorescence Cytometry. L. O. Henderson, G. E. Marti, A. Gaigalas, W. H. Hannon, R. F. Vogt Jr., Cytometry, 33:97-105, 1998.

14. Antiphospholipid antibody binding to bilayer-coated glass microspheres. A. R. Obringer, N. S. Rote, A. Walter, J. Immunol. Methods, 185 1, 81-93, 1995.

15. Ca2+ concentration during binding determines the manner in which annexin V binds to membrane. P. J. Trotter, M. A. Orchard, J. H. Walker. Biochem J., 308, 591-8, 1995.

1. A monoreagent for detecting and quantifying platelet-derived microparticles (PMPs) in a blood sample by flow cytometry, comprising:
a) a reagent 1 for double labeling the PMPs, comprising:
either annexin V, or any other marker specific for membrane phospholipids, coupled to a fluorochrome 1,
or a population 1 of MABs, directed against platelet membrane structures, labeled with a fluorochrome 1, (reagent 1a), and
a population 2 of monoclonal antibodies (MABs), directed against platelet membrane structures, labeled with a fluorochrome 2 (reagent 1b), and,
b) a reagent 2 consisting of a mixture of microspheres comprising:
a population of microspheres A labeled with one of the two fluorochromes 1 and 2, or another fluorochrome with a spectrum similar to one of the two, used to define the region of analysis of the PMPs in terms of size (light scattering parameters) and allowing differentiation thereof (setting beads),
a population of microspheres B, unlabeled or labeled with one of the two fluorochromes 1 and 2, or another fluorochrome with a spectrum similar to one of the two, the concentration of which is known, used as internal standard for counting the microparticles (counting beads),
a population of microspheres C labeled only with fluorochrome 1, used to define, with respect to the fluorochrome 1 fluorescence parameter, the minimum intensity threshold which delimits the region of analysis of the microparticles to which the MABs 1 labeled with fluorochrome 1 or the annexin V, or any other marker specific for membrane phospholipids. labeled with fluorochrome 1, are bound (threshold beads),
and a population of microspheres D, of diameter identical to C, and labeled only with fluorochrome 2, used to define, with respect to the fluorochrome 2 fluorescence parameter, the minimum intensity threshold which delimits the region of analysis of the microparticles to which the MABs 2 are bound (threshold beads).

2. The monoreagent as claimed in claim 1, characterized in that each population C and D of threshold beads is composed of two subpopulations of beads with two levels.

3. The monoreagent as claimed in claim 1, characterized in that the counting beads are greater than or equal to 5 μm in diameter.

4. The monoreagent as claimed in claim 1, characterized in that the counting beads are between 5 and 10 μm in diameter.

5. The monoreagent as claimed in claim 1, characterized in that the setting beads are between 1 and 0.5 μm in diameter.

6. The monoreagent as claimed in claim 1, characterized in that the threshold beads are 3 μm in diameter.

7. The monoreagent as claimed in claim 1, characterized in that the monoclonal antibodies (MABs) of the populations 1 and 2 are directed against platelet membrane structures chosen from the following specificities: CD61, CD41, CD42a, CD42b, CD42c, CD49b, CD29, CD62P, CD63, protein S and prothrombin.

8. The monoreagent as claimed in claim 1, characterized in that the populations 1 and 2 of MABs consist of a single MAB, of several MABs with the same specificity but directed against different epitopes, or of several MABs with different platelet specificities.

9. The monoreagent as claimed in claim 1, characterized in that reagent 1 of the monoreagent of the invention consists of annexin V, or another marker specific for membrane phospholipids, labeled with a fluorochrome 1 (reagent 1a), and of a population 2 of MABs labeled with a fluorochrome 2 (reagent 1b).

10. The monoreagent as claimed in claim 1, characterized in that reagent 1 consists of annexin V labeled with a fluorochrome 1 and of a population 2 of MABs labeled with a fluorochrome 2.

11. The monoreagent as claimed in claim 10, characterized in that the population 2 of MABs consists of several MABs with different specificities.

12. The monoreagent as claimed in claim 10, characterized in that the population 2 of MABs consists of anti-CD61 MABs and anti-CD42b MABs.

13. The monoreagent as claimed in claim 10, characterized in that the population 2 consists of the MABs P18 and 4F8 (anti-CD61) and SZ2 (anti-CD42b).

14. The monoreagent as claimed in one of claims 1 to 13, characterized in that fluorochrome 1 is FITC and fluorochrome 2 is PE.

15. The monoreagent as claimed in one of claims 1 to 14, characterized in that the sample is whole blood.

16. A diagnostic kit for detecting and quantifying PMPS, characterized in that it comprises a monoreagent as claimed in any one of claims 1 to 15.

17. The kit as claimed in claim 16, characterized in that it also comprises a dilution buffer.

18. The kit as claimed in claim 16, characterized in that it comprises:
a reagent 1a consisting of a population 1 of MABs specific for platelet membrane structures, or annexin V or any other marker specific for membrane phospholipids, labeled with a fluorochrome 1,
a reagent 1b consisting of a population 2 of MABs specific for platelet membrane structures, labeled with a fluorochrome 2,
a reagent 2 consisting of:
a population of beads for setting the analysis window for the PMPs, labeled with fluorochrome 1 or 2, or another fluorochrome with a spectrum similar to one of the two,
a population of counting beads, unlabeled or labeled with fluorochrome 1 or 2, or another fluorochrome with a spectrum similar to one of the two,
a population of threshold beads, preferentially consisting of two subpopulations of beads with two levels, labeled with fluorochrome 1,
a population of threshold beads, preferentially consisting of two subpopulations of beads with two levels, labeled with fluorochrome 2,
a reagent 3 consisting of a dilution buffer.

19. The kit as claimed in one of claims 16 to 18, characterized in that reagent 1a consists of annexin V and reagent 1b consists of a mixture of MABs with different specificities.

20. The kit as claimed in one of claims 16 to 18, characterized in that reagent 1b consists of a mixture of anti-CD61 and anti-CD42b MABs.

21. A method for detecting and quantifying PMPs, characterized in that it comprises the following steps:
a) bringing a blood sample into contact with a monoreagent as claimed in any one of claims 1 to 15 and incubating them, so as to obtain double color labeling of said PMPs, and
b) cytometrically analyzing the double labeled events.

22. The use of a monoreagent as claimed in any one of claims 1 to 15, or of a diagnostic kit as claimed in any one of claims 16 to 20, in a method for detecting and monitoring a prothrombotic condition.
